(19) United States
(12) Patent Application Publication
(10) Pub. No.: US 2023/0170086 A1
(43) Pub. Date: Jun. 1, 2023

(54) HEALTH MONITORING OF A PATIENT VIA GEOFENCING

(71) Applicant: Medtronic, Inc., Minneapolis, MN (US)

(72) Inventors: Eric Daniel James Dorphy, Maple Grove, MN (US); Tony J. Riley, Minneapolis, MN (US); Matthew R. Yoder, Crystal, MN (US)

(21) Appl. No.: 18/057,576

(22) Filed: Nov. 21, 2022

Related U.S. Application Data
(60) Provisional application No. 63/284,741, filed on Dec. 1, 2021.

(51) Int. Cl. G16H 40/67; G16H 50/20

(52) U.S. Cl. CPC G16H 40/67; G16H 50/20; A61B 5/0015

(57) ABSTRACT

This disclosure is directed to medical systems and techniques for enhanced health monitoring using geofencing. In one example, a patient may operate a computing device that is programmed to determine that a current patient location corresponds to a designated area of a medical system in response to receiving, by an input device, input data comprising an indication of the current patient location; in response to the indication, generate output data comprising one or more datasets of patient data for a data submission to a health monitoring service, wherein the patient data is stored in memory for a patient having a medical device configured to generate at least a portion of the patient data; and by an output device, transmit, to the health monitoring service, the output data to complete the data submission.

This application claims the benefit of U.S. Provisional Application Ser. No. 63/284,741, filed 1 Dec. 2021, the entire contents of which is incorporated herein by reference.

### FIELD

The disclosure relates generally to medical systems and, more particularly, medical systems configured to monitor patient activity for changes in patient health and/or to deliver therapy to a patient.

### BACKGROUND

Some types of medical systems may monitor various types of data of a patient or a group of patients for one or several purposes. Amongst the numerous examples, some medical systems may record measurements of a patient and their heart as indicia of cardiac health for that patient, which may be memorialized in raw data and/or processed data formats; as one example, electric signals representing cardiac activity over a period of time may be memorialized as a cardiac electrogram (EGM) and then, processed into other indicia of the cardiac health of the patient. In some examples, the medical system may monitor the EGM to detect one or more types of arrhythmia, such as bradycardia, tachycardia, fibrillation, or asystole (e.g., caused by sinus pause or AV block). In some examples, the medical system may include one or more of an implantable medical device or a wearable device to collect various measurements used to detect changes in patient cardiac health. In some examples, the medical system may deliver a therapy, and may record information regarding the delivery of therapy.

### SUMMARY

Medical systems and techniques as described herein facilitate patient administration of their medical care while continuing to monitor patient activity and detect changes in patient health (if any). The present disclosure describes “medical care” as referring to any product and/or process capable of making a patient more healthful and, typically, includes medical procedures, clinical evaluations, methods for providing therapy, methods involving diagnosis and/or treatment, healthcare-related processes, and/or the like. Administration of “medical care” for any given patient generally involves providing medical personnel with sufficient patient data for successfully performing the medical process and/or delivering the medical product.

Current methodologies for the administration of the patient's medical care have inefficiencies and/or latencies, possibly jeopardizing the patient's health. The inefficiencies and/or latencies may cause transmissions of patient data from the patient's devices at very low speeds or to be dropped entirely. This may result in the available patient data for medical personnel to be unreliable, inaccurate, and/or incomplete. Moreover, obtaining sufficient patient data may become time-consuming, difficult, or impossible. Opportunities are missed where recent patient data may be easily acquired and then, used to facilitate the administration of the patient's medical care.

Human activity and a lack of tools prevent coordination or automation of at least a portion of the current methodologies described above. There may be tools for certain operations, but those tools are restricted in terms of capability, for example, relying upon the patient's personal device to perform adequately. If the patient's personal device is shut down, not running a particular application, running a particular application in a background, or is without network connectivity, the above-mentioned tools are limited in their operation and, most likely, cannot transition to a next step in the administration, for example, of a clinical examination for the patient.

Acknowledging the lack of tools for administering, with adequate efficiency, the medical care for the patient, the present disclosure describes medical systems configured to enable automatic activation of certain actions by the patient's device(s) including transmissions of information corresponding to a health of the patient. The present disclosure further describes, for these medical systems, techniques that ensure consistency and sufficiency in the patient data stored at an external location. The techniques of the present disclosure allow the medical systems described herein to manage latencies corresponding to uploads of current patient data stored at a patient device, a medical facility computing system, and/or a device of a clinician/medical specialist treating the patient, for example, by reducing delays from a time of recording (e.g., at a patient device, at a medical device, and so forth) to a time of transmission (e.g., to the health monitoring service, to medical systems, and so forth).

The above-mentioned delays and inefficiencies may result in misused resource capacities and waste considerable amounts of time and capital. In general, these issues frustrate the health monitoring service as described herein by interfering with its programming for performing a patient data assessment, a device interrogation, and health event (e.g., arrhythmia) detection. Without sufficient patient data at a time when a medical specialist needs that data, a proper assessment cannot be done. The medical systems and techniques may accomplish at least a proximate improvement in patient physical/mental health, especially considering that the above-mentioned issues can result in less efficacious medical care for the patient. Furthermore, by reducing the delays and inefficiencies, the medical systems and techniques of the present disclosure may achieve multiple goals, such as lowering resource requirements, lowering resource utilization, and improving upon an overall cost. In view of the above, the present disclosure describes a technological improvement or a technical solution that is integrated into a practical application.

To illustrate by way of an example medical system, a computing system for running, over a network, a computing service (e.g., a cloud computing service), referred to herein as a health monitoring service, may be configured to maintain recent and up-to-date patient data in network accessible storage resources. Generally, when the health monitoring service requires and/or requests a dataset of patient data or other information stored in the patient's medical device, the patient's medical device attempts to fulfil that requirement by communicating the requested dataset. If, for some reason, the patient's medical device fails to transmit the dataset or a transmission of the dataset fails, for instance, becomes delayed or corrupted, the health monitoring service may avail a proximate device for prompting a data submission of the requested dataset. According to at least one technique, the health monitoring service may use geofencing to complete any patient data submission that is delayed, terminated, or otherwise, inappropriate, for instance, by forcing the patient's medical device to complete any missed or pending transmissions of requested patient data. The health monitoring service may leverage a proximate device in communication with the patient's medical device for initiating actions related to completing missed or pending transmissions of the patient data.

For the patient's personal device, a device of a clinician/medical specialist treating the patient, or another computing device that is different from the patient's medical device, the health monitoring service may enable a remote connection through which submissions of current patient data arrive at an interface to the storage resources. The interface handles each upload by extracting the current patient data and updating a local copy of the patient data. The health monitoring service may configure a device to operate as a beacon device configured to relay messages to the patient's medical device; examples of these messages are described herein and prompt the medical device to perform various actions. A number of these actions are directed to complete the data submissions under certain conditions.

An example data submission may be an unscheduled memory uplink interrogation. The health monitoring service, via the beacon device, may submit a request (message) identifying the interrogation to initiate and providing a set of parameters for appropriate submissions. Instead of a scheduled timeslot parameter, the request may indicate an expected date/time parameter and/or a last acceptable data/time parameter. For a number of reasons, the data submission may arrive late/corrupted, or the data submission somehow stopped/faulted unexpectedly. According to at least one technique, the health monitoring service may communicate a message to the beacon device, which relays the message to the patient's medical device in order to resume the unscheduled memory uplink interrogation after a delay and before the current patient data to be interrogated becomes stale. Upon receiving the message, the patient's medical device may determine that the request is past due, the expected date/time parameter is not satisfied, and the requested patient data is now late. The unscheduled interrogation may be considered late for missing a latest transmission time. If the requested patient data can still be used to the benefit of the health monitoring service, the patient's medical device may generate output data comprising the one or more datasets of the patient data corresponding to the past due request.

Another example data submission may be a scheduled memory uplink interrogation. The health monitoring service, via the beacon device, may provide a set of parameters for appropriate scheduled data submissions. At least one parameter may establish a schedule with patient medical devices where timeslot(s) are assigned for their respective data submission(s). The scheduled data submission may become pending, late, and/or interrupted, for example, if the schedule timeslot is elapsed by a current time. The health monitoring service, via the beacon device, may relay a message identifying the scheduled memory uplink interrogation to be resumed/restarted/rectified. The patient's medical device, upon receiving the message to facilitate competition of the scheduled memory uplink interrogation. In accordance with the schedule stored in memory, the patient's medical device may determine that the scheduled timeslot has been missed and requires restart. The message may cause the patient's medical device to generate output data comprising the one or more datasets of the patient data for the data submission that corresponds to the scheduled timeslot.

In one example, a medical system comprises: processing circuitry executing logic stored in memory configured to: detect a presence of a patient device within a designated area based on a broadcast from the patient device; based on the broadcast, determine that the patient device comprises a monitoring application configured to upload, to a health monitoring service operating on a computing system within the designated area of the medical system or from an external location of the medical system, datasets of patient data, wherein a portion of the patient data comprises interrogation data of a medical device for a patient; communicate a message comprising an indication of a current patient location, wherein the message is configured to trigger one or more automatic data submissions by the monitoring application as a response to receiving the message; and receive, from the patient device, the one or more automatic data submissions for the health monitoring service.

In another example, a method comprises determining, by the processing circuitry, that a current patient location corresponds to a designated area of a medical system in response to receiving, by an input device, input data comprising an indication of the current patient location; in response to the determination, generating, by the processing circuitry, output data comprising datasets of patient data for a data submission to a health monitoring service, wherein the patient data is stored in memory for a patient having a medical device configured to generate at least a portion of the patient data; and by an output device, transmitting, to the health monitoring service, the output data to complete the data submission.

In another example, a non-transitory computer-readable storage medium comprises program instructions that, when executed by processing circuitry of a medical system, cause the processing circuitry to: detect a presence of a patient device within a designated area based on a broadcast from the patient device; based on the broadcast, determine that the patient device comprises a monitoring application configured to upload, to a health monitoring service operating on a computing system within the designated area of the medical system or from an external location of the medical system, datasets of patient data, wherein a portion of the patient data comprises interrogation data of a medical device for a patient; communicate a message comprising an indication of a current patient location, wherein the message is configured to trigger one or more automatic data submissions by the monitoring application as a response to receiving the message; and receive, from the patient device, the one or more automatic data submissions for the health monitoring service.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a block diagram illustrating an example configuration of a medical device, in accordance with one or more examples of the present disclosure.

FIG. 3 is a block diagram illustrating an example configuration of the external device of FIG. 1, in accordance with one or more examples of the present disclosure.

FIG. 4 is a block diagram illustrating an example architecture of the health monitoring service of FIG. 1 that is operative on the computing system of FIG. 1, which may be coupled to the medical device and external device of FIGS. 1-4, in accordance with one or more examples of the present disclosure.

FIG. 5 is a flow diagram illustrating an example operation for administering medical care in a geofenced geographic area, in accordance with one or more examples of the present disclosure.

Like reference characters denote like elements throughout the description and figures.

### DETAILED DESCRIPTION

In general, medical systems according to this disclosure implement techniques for using geofencing to enhance a health monitoring service with improved effectiveness. The present disclosure describes the health monitoring service as a computing service operative on a computing system. A number of computing devices may execute and run the health monitoring service in multiple locations connected by networking infrastructure.

Proper administration of a patient's medical care depends on availability and utility of up-to-date patient data. As described herein, the health monitoring service may use patient data for a number of reasons and, to a certain extent, depends on having sufficient patient data in order for certain operations to succeed. The health monitoring service allows medical personnel to securely retrieve up-to-date patient data, directly or indirectly, from the patient device and/or from an external storage location. The medical personnel at a clinic further benefit from the availability of the up-to-date patient data provided by the health monitoring service. To this end, the patient may program a patient device to automate submissions of patient data to devices in the designated area, ultimately reaching a local computing system for a local server of the clinic and/or the (remote) computing system for operating the health monitoring service. However, for a number of reasons (e.g., lack of network connectivity, inoperable device, non-functioning mobile application, and/or the like), the health monitoring service and/or the local (clinic) server the patient may be unable to exchange all necessary data.

To overcome the above reasons, the medical systems and techniques described herein may enforce (e.g., overdue) data submissions by way of rules prescribing under which condition(s) datasets of patient data are to be transmitted by wire or wirelessly over a communication connection with those non-patient device(s). According to one example technique, the health monitoring service may designate a geographic area as an available and secure environment in which a computing device of the health monitoring service facilitates the medical care for the patient. When a patient enters the designated area to receive medical care, the health monitoring service may direct a patient device to perform one or more operations to facilitate the administration of that medical care. In some examples, the patient device may automatically perform the above one or more operations on behalf of the patient to their benefit.

The patient may have a patient device (e.g., a mobile device) in addition to their medical device. Even when the patient device is connected to the health monitoring service by way of a communicative coupling, the patient device may fail to transmit (e.g., upload) the patient data desired or required by the health monitoring service. The patient device and the health monitoring service may be connected to each other for a considerable amount of time but also neglect advantageous times to submit or transmit the desired patient data. The patient device may execute and run a monitoring application for coordinating patient data submissions to the health monitoring service (e.g., based on interrogation data from the medical device).

However, there may be instances where the monitoring application is inhibited or unable to perform such coordination. For example, the patient device may impose hardware/software restrictions preventing the monitoring application from communicating with the medical device to retrieve the desired patient data and/or with the health monitoring service to transmit the desired patient data. This restriction may be set as a content restriction by an operating system of the patient device. In other examples, the patient device (e.g., via the operating system) may have the monitoring application shut down, deactivated (e.g., paused), confined to run in as a background application, and/or is otherwise unable to communicate with the medical device or health monitoring service. Even when the monitoring application is active/operational (e.g., running in the background), failing to recognize opportune situations for retrieving (e.g., from the medical device) and then, transmitting the desired patient data often still occur.

Medical systems and techniques capable of availing these opportunities to benefit the patient and the administration of their medical care, for example, by ensuring an up-to-date copy of the patient data is accessible at a time of the patient is undergoing a medical procedure. The monitoring application may achieve up-to-date copy may be accomplished by collecting required (e.g., recent) information (e.g., sensed physiological information, device interrogation data, and/or the like) in preparation for the patient receiving medical care.

Medical systems and techniques capable of reducing or eliminating above-mentioned delays and inefficiencies improves patient care, for instance, by increasing the likelihood that the patient's clinician has up-to-date and complete information at a clinic visit. With respect to these examples, the clinician would probably prefer having their up-to-date copy of the patient data before the clinic visit but there may be instances where complete and/or up-to-date patient data is not available until the patient arrives at the clinic (e.g., enters the geofenced area associated with the clinic); for these situations, the present disclosure describes at least one medical system and technique for triggering a patient device to (e.g., automatically without further user input) transmit, to a device of a health monitoring service, datasets of (e.g., recent and/or historical) patient data. The clinician, in one example, may configure a local computing device operating within the clinic to detect a presence of the patient device upon entering the geofenced area. When the patient brings their patient device to the clinic visit, the local computing device, in response to detecting the patient device, communicates, to the patient device, a message indicative of a current patient location, prompting the patient device (e.g., using a monitoring application) to automatically perform an interrogation operation to retrieve interrogation data from the medical device and then, transmit, to the local computing device, the retrieved interrogation data to update the clinic copy of the patient data. In some examples, the interrogation data may include the most recent patient data or data from any other periods of time. The local computing device may configure another device to operate as an intermediate between the patient device and itself. For the health monitoring service described above, the present disclosure describes a number of embodiments of which some examples may utilize a programmable beacon to detect the presence of the patient and/or communicate the indication of the current patient location.

The present disclosure further describes the health monitoring service as a computing service for enabling certain functionality of the patient device, for example, for completing (e.g., restarting) past due submissions of patient data or commencing same or similar data submissions that are currently due. Therefore, having secure, reliable, and immediate access to the health monitoring service facilitates provision (e.g., delivery) of medical care to the patient from a geofenced area designed by the service for administering the patient's healthcare needs. Having at least one device in the geofenced area communicatively coupled to the health monitoring service enables functionality of the health monitoring service without requiring medical personnel be present or a private room for patient observation. For instance, the patient and their cardiologist do not need to meet in a private room at the facility for an interrogation of the patient's medical device. This is (in part) because the patient's home or the above-mentioned medical facility are geofenced areas providing an immediate connection for transmissions of private data from that medical device to a device of the health monitoring service. Therefore, the health monitoring service may initiate the device interrogation for the patient's benefit and while the patient remains located in the geofenced area (e.g., a lobby of the emergency room, hospital, clinic, or any other medical facility).

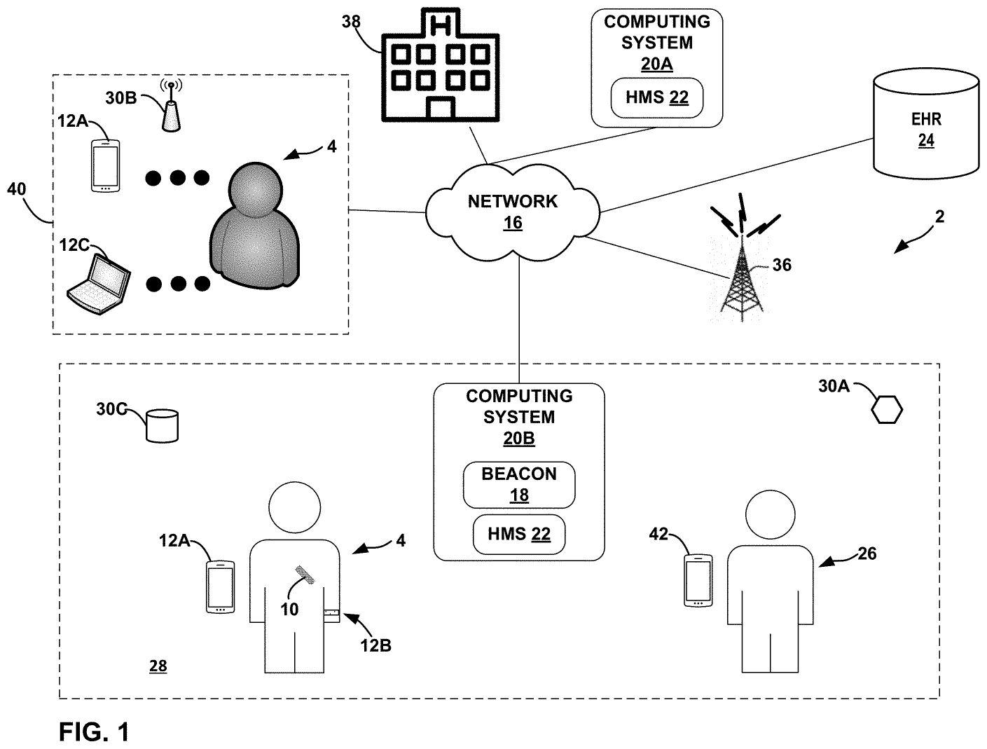

FIG. 1 is a block diagram illustrating an example system 2 configured detect health events of a patient 4, and to respond to such detection, in accordance with one or more techniques of this disclosure. As used herein, the terms “detect,” “detection,” and the like may refer to detection of a health event presently (at the time the data is collected) being experienced by patient 4, as well as detection based on the data that the condition of patient 4 is such that they have a suprathreshold likelihood of experiencing the health event within a particular timeframe. Examples of acute health events are a cardiac arrest, a ventricular fibrillation, a ventricular tachycardia, a stroke, a seizure, or a fall. The example techniques may be used with one or more patient sensing devices, e.g., implantable medical device (IMD) 10, which may be in wireless communication with one or more patient computing devices, e.g., patient computing devices 12A and/or 12B (herein “patient computing devices 12”). Although not illustrated in FIG. 1, IMD 10 may include electrodes and/or other sensors to sense physiological signals of patient 4, and may collect and store sensed physiological data based on the signals and detect episodes based on the data.

In some examples, IMD 10 may be implanted outside of a thoracic cavity of patient 4 (e.g., subcutaneously in the pectoral location illustrated in FIG. 1). In some other examples, IMD 10 may be implanted at other subcutaneous locations on patient 108 such as at an abdominal location. IMD 10 may be implanted subcutaneously or submuscularly on the left midaxillary of patient 4, such that IMD 10 may be positioned on the left side of patient 4 above the ribcage.

IMD 10 may be positioned near the sternum near or just below the level of the heart of patient 4, e.g., at least partially within the cardiac silhouette. In some examples, IMD 10 takes the form of the LINQ™ insertable cardiac monitor (ICM). Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted patient sensing device, in some examples a system includes one or more patient sensing devices, which may be implanted within patient 4 or external to (e.g., worn by) patient 4.

Various device(s), including computing devices 12, external (non-patient) computing devices (such as those for computing system 20A and/or computing system 20B), Internet of Things (IoT) devices 30, among other devices illustrated in FIG. 1 for area 28, may be configured to communicate with IMD 10, for example, via wireless network connectivity (e.g., a local area network or the Internet). The above-mentioned device and, possibly, other devices may be communicatively coupled to IMD 10 for performing one or more operations, such as a device interrogation operation to retrieve interrogation data. To receive the interrogation data and, possibly, other patient data, a number of the above-mentioned devices may be communicatively coupled to any of computing device(s) 12. As an option, another computing device (not illustrated in FIG. 1) may be connected to IMB 10 via a communicatively coupled and initiate, by way of that connection, a transmission of the patient data stored in memory of IMD 10 and/or computing device(s) 12.

Patient computing devices 12 are configured for wireless communication over a network with various devices including IMD 10, another patient computing device 12, computing systems 20, IoT devices 30, and/or the like. Computing devices 12 retrieve various types of information including datasets of patient data, such as event data (e.g., health events and/or alerts), monitored medical device data, and sensed physiological information that was collected and stored by IMB 10 and/or HMS 22. In some examples, a client for a (e.g., cloud) computing service referred to herein as HMS 22 may be configured to run, over the network, on computing system(s) 20 to store some of the above-mentioned types of patient data and/or additional types of information. HMS 22 may maintain, in an external location, an up-to-date copy of patient data (e.g., data from IMD 10); to ensure the copy is an accurate representation and further includes most recent datasets, HMS 22 may be configured to update that copy with datasets after receiving each data submission. For each data submission to HMS 22, computing system(s) 20 may be configured to receive (e.g., by triggering automatic transmissions of) patient data from IMB 10 and/or patient device(s) 12 for storage and subsequent use in updating a copy of the patient data maintained by the HMS 22.

In general, computing devices 12 that may be any computing device configured to run an application that enables the computing device to interact with IMD 10. In some examples, computing devices 12 take the form of personal computing devices of patient 4. For example, computing device 12A may take the form of a smartphone of patient 4, computing device 12B may take the form of a smartwatch or other smart apparel of patient 4, and computing device 12C may take the form of any personal computer configured for wireless communication with IMB 10, such as a desktop, laptop, a notebook computer, tablet computer, workstation, one or more servers, cellular phone, or personal digital assistant. One or more of computing devices 12 may be configured to communicate with one or more computing systems, e.g., computing systems 20A and 20B (collectively, “computing systems 20”) via network 16.

Computing devices 12 may communicate with IMD 10 and/or each other according to the Bluetooth® or Bluetooth® Low Energy (BLE) protocols, as examples. In some examples, only one of computing devices 12, e.g., computing device 12A, is configured for communication with IMD 10, e.g., due to operation of hardware (e.g., as part of a BLE receiver/transmitter module) and/or due to execution of software (e.g., part of a health monitoring application as described herein) enabling communication and interaction with an IMD 10. Computing devices 12 may communicate with any of the other devices described herein via near-field communication (NFC) technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

In some examples, computing device(s) 12, e.g., wearable computing device 12B in the example illustrated by FIG. 1, may include electrodes and other sensors to sense physiological signals of patient 4, and may collect and store physiological information and detect episodes and other health events based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A.

Computing systems 20A and 20B may be respectively managed by manufacturers of IMD 10 and/or computing devices 12 to, for example, provide cloud storage and analysis of collected data, maintenance and software services, or other networked functionality for their respective devices and users thereof.

Computing system 20A may comprise, or may be implemented by, the Medtronic CareLink™ Network, in some examples. In the example illustrated by FIG. 1, computing system 20A implements a health monitoring service (HMS) 22, although in other examples, either or both of computing systems 20 may implement HMS 22. As will be described in greater detail below, HMS 22 may communicate a message instructing IMD 10 on subsequent operation, for example, under certain conditions, such as when the above application is closed, inoperative (e.g., not running or closed), malfunctioning (e.g., false alarm), or otherwise is unable/unequipped to detect the false positive.

IMD 10 may generate an alarm (e.g., an audible and/or tactile alarm) to get the immediate attention of patient 4 or clinician 26 of a potential health event and at a later point-in-time, receive, from computing system 20A, a message directing IMD 10 to perform an action. In one example, computing system 20A may communicate the message to computing device 12A or IoT device 30A, causing in either device subsequent communication of the message to IMD 10, which prompts an application running in IMD 10 to output datasets of patient data corresponding to the potential health event.

HMS 22 may determine that a potential health event is a false positive and communicate a message instructing IMD 10 to silence or cancel an alarm (e.g., the audible and/or tactile alarm) for the potential health event. There may be cancellation criterion for the potential health event to satisfy in order to qualify the alarm for cancellation. Instead of or in addition to the alarm, IMD 10 may broadcast an alert message to any receiver within a certain proximity upon detecting the potential health event. The detection of the potential health event by IMD 10 may be in response to results based on an analysis of current patient data (e.g., recently sensed physiological signals of patient 4). HMS 22 may predict an alert and/or an alarm corresponds to a false detection by IMD 10 and then, communicate a message instructing IMD 10 to withhold transmission of any output datasets indicating the false detection of a potential health event, in effect, preventing the alert and/or the alert.

If a potential health event is a true positive, HMS 22 may communicate a message instructing IMD 10 to release a pending transmission of the output datasets comprising the health event. Alternatively, computing system 20B may receive an indication of an alarm and/or an alert message of the potential health event and then, bypassing IMD 10 or device 12A, complete the pending transmission from local storage resources on behalf of IMD 10 and/or device 12. HMS 22 may detect an error in IMD 10 based on the indication of an alarm and/or an alert message and communicate a message instructing IMD 10 to withhold transmission of future output datasets, in effect, halting operation of monitoring and detection functionality by IMD 10.

HMS 22 is an example of a computing service that provides additional storage, processing, and network resources to computing device(s) 12 and any other external device to IMD 10. HMS 22 may be a cloud (computing) service that provides computing resources (e.g., capacities and capabilities) over network 16. A network service may manage interactivity between HMS 22 and IMD 10, for example, directing communications to the intended recipients by enforcing a communication protocol on senders.

As described herein, HMS 22 may generate a geofence to prompt communications between HMS 22 and patient 4 or clinician 26 in geographic area(s) 28. Area 28 includes computing facilities, e.g., a local network, by which computing devices 12, IoT devices 30, and other devices within area 28 may communicate via network 16, e.g., with HMS 22. For example, area 28 may be configured with wireless technology, such as 802.11 wireless networks, 802.15 ZigBee networks, or the like. Area 28 may include one or more wireless access points that provide support for wireless communications throughout area 28. Additionally or alternatively, e.g., when local network is unavailable, computing devices 12, IoT devices 30, and other devices within area 28 may be configured to communicate with network 16, e.g., with HMS 22, via a cellular base station 36 and a cellular network.

Area 28 may be defined as a set of geographic coordinates within which a home, office, or place of business, or public venue, or medical facility, as examples, provide a secure environment for administrating medical care for patient 4. Area 28 may be where a type of medical facility (e.g., a clinic) is located to facilitate the local health monitoring service and/or the remote health monitoring service. Under direction of the medical system administering the local health monitoring service and/or the remote health monitoring service, clinician 26 may facilitate one or both services by performing medical procedures on different patients, including patient 4 and other patients.

Patient 4 visits area 28 to receive medical care as described herein, for example, including evaluations of the condition of patient 4 and IMD 10, or to undergo medical procedures. Medical systems may have designated hospital 38 and other medical facilities external to area 28 for HMS 22. If needed, a computing device in hospital 38 may retrieve an up-to-date copy of patient data from HMS 22. Because of the example techniques described herein, hospital 38 can be assured the retrieved copy of patient data represents most recent data of patient 4.

In some examples, patient 4 may receive medical care while at a non-medical facility 40, such as their home, office, or retirement community. Combined with network 16, HMS 22 enables computing device 12A to electronically interrogate IMD 10 while patient 4 is currently home, which may be represented in FIG. 1 as area 40, and then, relay the device interrogation data to HMS 22. Computing device 12A may communicate the device interrogation data to a central server that operates in a same location (as a part of a local computing system) or in an external location (as a part of a remote computing system). HMS 22 may configure computing device(s) 12 to automate data submissions for patient 4, for example, during opportunities to exchange patient responses to clinician queries, especially when patient 4 is at home at area 40 and not in area 28. While patient 4 is home, computing device 12C may be configured to submit patient data for any missed transmissions. The submitted patient data can then be viewed by the patient's physician and other personnel without the patient having to be present. Medical system 2 allows clinics to keep up with increasing demands for patient assessment and/or device interrogation while maintaining/improving patient safety (by accurate diagnosis of arrhythmias and device abnormalities) and convenience (by reducing or eliminating the number of trips to physician's offices). For example, after device interrogation from the patient's home, HMS 22 may initiate a remote review by a cardiologist. Similarly, HMS 22 may automatically connect computing device 12A to the cardiologist's device as patient 4 visits the emergency room, clinic, or other medical facility without the cardiologist being physically present in the same facility and/or an actual appointment, resulting in shorter wait times due to an early discharge/end of visit. This may be beneficial when patient 4 is having acute health event and in need of medical care.

Computing device(s) 12 may be configured with appropriate hardware/software component(s). HMS 22 may direct computing system 20A (or computing system 20B) to control a hardware/software component of computing device(s) 12 into transmitting datasets of patient data. HMS 22 may program the component to automate the above transmissions, for instance, when patient 4 is in a clinical setting for area 28. For instance, computing device 12A may include a transmitter that can interrogate IMD 10 and by way of that transmitter, have interrogated device data transmitted to HMS 22 via a connection that is either wireless or wired such that the data can be reviewed remotely by physicians, device clinic personnel, and device manufacturer personnel.

In some examples, processing circuitry in a wearable device, e.g., wearable computing device 12B, or a laptop computer, e.g., personal computing device 12C, may execute same or similar logic as the logic executed by processing circuitry of IMD 10, processing circuitry of computing device 12A, and/or other processing circuitry as described herein. As an alternative, computing device 12C may be proprietary device provided by a manufacturer of IMD 10. In this manner, a wearable device, a laptop computing, and/or other device may perform some or all of the techniques described herein in the same manner described herein with respect to IMD 10. In some examples, the wearable device operates with IMD 10 and/or computing device 12A as potential providers of computing/storage resources and sensors for monitoring patient activity and other patient parameters. For example, the wearable device may communicate the patient data to computing device 12A or 12B, computing system 20A or 20B, and/or IoT devices 30A, 30B, or 30C for storage in non-volatile memory and for computing parameter values or metric values from patient physiological measurements and other sensed patient data.

Computing device(s) 12 may store in memory and/or transmit via a network interface various types of information as described herein. Examples of the types of data managed by computing device(s) 12 may include sensed data, e.g., values of physiological parameters measured by IMD 10 and, in some cases one or more of computing devices 12, data regarding episodes of arrhythmia or other health events detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect health events for patient 4. In some examples, HMS 22 provides data from EHR 24 to computing device(s) 12 and/or IMD 10 for storage therein and use as part of their algorithms for detecting health events.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

In any of the examples described herein, receiving input data may prompt an application (e.g., a client application) running in IMD 10 and/or computing devices(s) 12 to complete past or currently due submissions of data (e.g., patient data) for HMS 22. HMS 22 may be operative within one or more geographic areas and confined therein such that patient 4 entering any one of these areas (i.e., a geofence) with computing device 12 causes activation, by another computing device, of the client application and/or engagement by patient 4, via computing device 12, with the health monitoring service. HMS 22 may prompt computing device(s) 12, via the client application, to perform one or more operations, thereby triggering one or more data submissions to the other computing device for storage. Some data submissions may be considered missed transactions because the client application failed to complete these data submissions for some reason (e.g., being shutdown).

There may be an examples where the above-mentioned client application is not running in computing device 12A for patient 4 and equivalent application logic is executed by and running in processing circuitry of another device within area 28 or another designated area. In one example, application logic running in computing device 12B (with or without any input from clinician 26) may be activated for patient 4 and used by HMS 22 to administer tasks for patient 4's medical care. The application logic may be activated to handle data submissions to HMS 22 in a manner similar to computing device 12A for patient 4. For example, computing device 12B may respond to HMS 22 performing tasks for (medical) device interrogation and/or patient assessment by uploading data from IMD 10 and/or downloading data from HMS 22 via network 16. The uploaded data may include timestamped datasets of sensed patient data, and the downloaded data may include documents (to sign and return), forms (to complete and submit), and updates (to load into processing circuitry and execute). The application logic may be the same logic for the monitoring application described herein and therefore, capable of being communicatively coupled to IMD 10. The application logic in computing device 12B may be different logic from the monitoring application. In other examples, without computing device 12A executing the monitoring application, the application logic may be executed to run in a background or foreground of computing device 12B as the monitoring application.

Area 28 may represent a geographic area that a medical system designates for health monitoring service(s) including HMS 22 to run on computing system(s) 20. HMS 22 may define a geofence for area 28 and use that geofence to protect from intrusion data transmissions to/from computing devices 12 when those devices 12 are physically present within area 28. In this manner, computing device 12A may be prompted to perform one or more actions; one example action is to complete past due and/or presently due data submissions to HMS 22, which may desire completion of each data submission for maintaining and/or improving upon a quality of the medical care provided to patient 4. The datasets of patient data being transmitted to computing system(s) 20 may be necessary and critical for HMS 22 to function properly.

Other devices in the area 28 of patient 4 may also be configured to take one or more actions with respect to patient 4 and, possibly, a clinician 26, or to otherwise facilitate the delivery of care to patient 4. For example, area 28 may include one or more IoT devices, such as IoT devices 30A-30C (collectively “IoT devices 30”) illustrated in the example of FIG. 1. IoT devices 30 may include, as examples, so called “smart” speakers, cameras, lights, locks, thermostats, appliances, actuators, controllers, or any other smart home (or building) devices. In the example of FIG. 1, IoT device 30C is a smart speaker and/or controller, which may include a display. A beacon device, an example embodiment for IoT devices 30, may provide functionality for HMS 22 and/or clinician 26. As described herein, the beacon device may communicate a message invoking a wake-up function call on IMD 10.

For the client application running in computing device 12A, a second and different application (e.g., a beacon application) running in computing device 12A may implement an API with functionality for activating appropriate logic (e.g., software code) for performing one or more operations including any given data submission, for example, by way of a function call on that API. Each data submission may involve computing device 12A performing the one or more operations including preparation of various datasets of patient data for submission to computing device 12B followed by transmission and then, completion of the above data submission(s). There are a number of example techniques for initiating any type of data submission, such as, for example, by configuring (application) logic with functionality that when invoked, triggers an automatic transmission (e.g., broadcast) by computing device 12A of output data to one or more receiving devices.

In addition to geofencing, HMS 22 may avail other controls and methods for monitoring the environment within area 28. Computing system 20B may configure a computing service on a local area network within area 28. A local server device may operate the computing service as a local service (e.g., via a radio access technology (RAT)). Logic (e.g., the monitoring application) for IMD 10 and/or computing device 12A may configure one or both device(s) to expose function calls via an API available only on the local area network. In response to receiving input data from a device of computing system 20B, computing device 12A may identify an indication of a function call on that API and responsive to that identification, initiate the example data submission as described herein.

A medical facility (e.g., a clinic) may operate computing system 20B as a resource in their provision of medical care. A device of computing system 20B may include processing circuitry configured with logic that, when executed, is operative to detect a presence of a patient device, such as or computing device(s) 12, within area 28 (i.e., a designated area) based on a broadcast message distributed by that patient device. As described herein, computing device(s) 12 include a monitoring application configured to upload one or more datasets of patient data to HMS 22 while running on computing system 20A at an external location of medical system 2. A portion (e.g., a dataset) of the patient data may include interrogation data of IMD 10 for patient 4.

While HMS 22 and an application running in IMD 10 and/or computing device(s) 12 may be configured to cooperate, for example, by exchanging data in a manner that is compatible with both software components, the medical system may not require the application to communicate over network 16 with HMS 22. Alternative medical systems may include separate sub-systems for HMS 22 and the application respectively. Hence, as HMS 22 launches a service (e.g., a cloud service) and has patient 4 brings their devices online with this service, computing device 12A may install and/or open the application to commence operating as a service agent, in some examples, or as a software component, incompatible with HMS 22, according to other examples. In general, the above-mentioned application is configured to initiate health event monitoring of various types of information, for example, by identifying, in a dataset for patient 4, indicia of interest regarding health (e.g., cardiac health) of patient 4.

Computing device(s) 12 and/or computing system(s) 20 may implement one or more algorithms in support of medical system 2 and its administration of health monitoring service(s) 22. The above-mentioned examples generate an API between the monitoring application and HMS 22; however, in other examples, that API is not necessary and HMS 22 may continue monitoring patient 4. One alternative to the examples depicted in FIG. 1 may configure the monitoring application to use network 16 (e.g., Internet) to communicate with computing system(s) 20. In some examples, the monitoring application is still operative to complete missed or pending transactions in examples where it cannot or does not communicate with another device over network 16; to illustrate, computing device 12A may include a Bluetooth® module to enable Bluetooth® telemetry with another computing device, such as computing system 20B.

HMS 22 may be configured to use additional technologies to trigger and then, retrieve submissions while operating in one or more geofenced geographic areas. HMS 22 may use BLE to remotely trigger operations by computing device(s) 12, such as the triggering of the above-mentioned device interrogations and/or transmissions, in the one or more geofenced geographic areas in order to secure the submissions of patient data from the patient device(s) and to capture such information at opportune times. While a normal operating mode is active, IMD 10 and/or computing device 12A may be programmed to upload datasets of patient data in accordance with a schedule. These data submissions may be performed a specific number of times per day (e.g., once a day, such as at midnight). The specific number may be capped to converse battery power and other reasons. HMS 22 enables an extra transmission of patient data to a device of HMS 22. The extra transmission may be warranted when patient is in certain geofenced areas, such as at a clinic for follow-up visit, or another medical facility for a different medical procedure.

The medical systems and techniques create hardware/software components to improve the efficiency of assessing patients with implantable medical devices. Because access to the physical programmers and trained personnel is limited, delays in device assessment are common, leading to inefficiencies in patient care. In some examples, the techniques of this disclosure make better use of the time of such professionals and equipment by collecting patient data prior to a clinic visit, e.g., in a waiting room of the clinic, rather than during the visit with the clinician. Emergency room visits often turn into requests to assess implantable medical devices in that setting, and the techniques described herein may facilitate such assessments in settings that do not necessarily include the trained personnel or programmers. Increasing healthcare costs have led to increased scrutiny of the device interrogation/patient assessment process by physicians, patients, device manufacturers, and health care organizations (hospitals, insurers, and governments). To overcome the increased costs, the medical systems and techniques of the present disclosure enable peri-procedural device interrogation/patient assessment over network 16 and, in some examples, may program computing device(s) 12 to automatically transmit datasets of patient data based on current patient location and depending on requirements of HMS 22. Computing device(s) 12 are now configured to control (e.g., schedule) submissions of patient data to HMS 22 without a human user. By removing human error in every upload of recent patient data, HMS 22 is able to keep up-to-date its copy of the patient data and avoid problems caused by human error.

The application of medical systems and techniques further reduces the time patients spend in high-cost areas and shorten the time device patients spend in different medical facilities, for example, after surgical procedures, during clinician visits, and/or the like. These improvements in efficiency may lead to improved quality of care for patients and reductions in healthcare costs for our medical system. In some clinical situations where patient assessment with a programmer or a specialist is required, the health monitoring service may provide more options for connectivity (e.g., via land line, Wi-Fi, and cellular).

While computing device(s) 12 may be communicatively coupled to IMD 10, it should be noted several alternatives to IMD 10 including multiple medical devices or no medical device. In some examples, patient 4 may have computing device 12 and IMD 10 as part of independent systems, and in at least example, patient 4 does not have a medical device implanted into their body. Because the patient's mobile device may allocate some of its resources for use with the patient's medical device, the medical system and the techniques described herein may configure the mobile device and the medical device to cooperate during medical device interrogation or patient data assessment, for instance, by operating on a same layer to the health monitoring service.

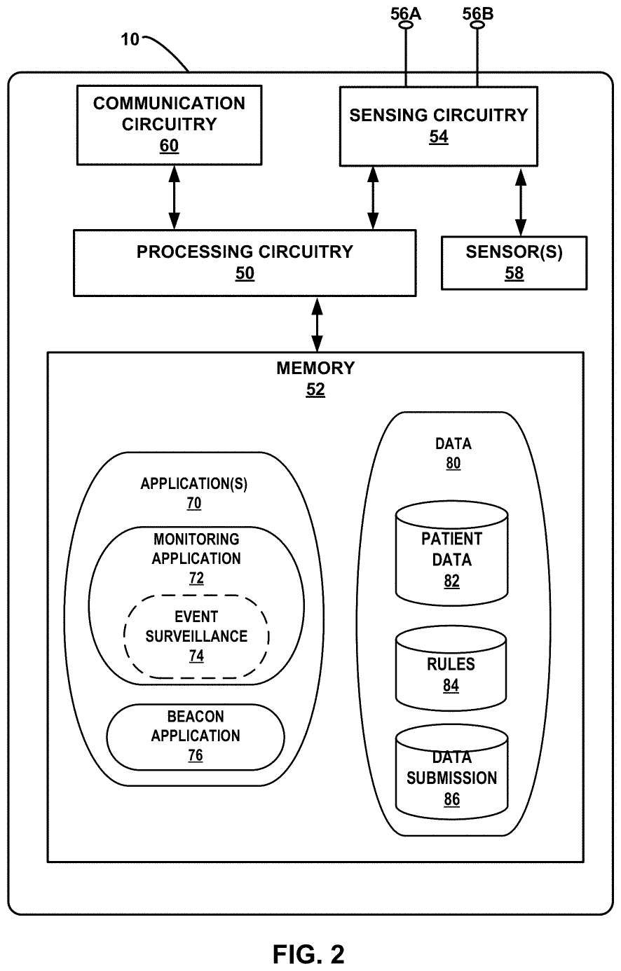

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured impedance.

In some examples, IMD 10 includes one or more sensors 58, such as one or more accelerometers, microphones, optical sensors, temperature sensors, and/or pressure sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications 70 executable by processing circuitry 50, and data 80. Applications 70 may include a monitoring application 72 and beacon application 76. Processing circuitry 50 may execute monitoring application 72, invoking event surveillance 74, to detect a health event of patient 4 based on combination of one or more of the types of physiological data described herein, which may be stored as sensed data 82. In some examples, sensed data 82 may additionally include data sensed by other devices, e.g., computing device(s) 12, and received via communication circuitry 60. Event surveillance 74 may be configured to operate with monitoring application 72 to apply rules 84 to sensed data 82. Rules 84 may include one or more models, algorithms, decision trees, and/or thresholds. In some cases, rules 84 may be developed based on machine learning.

Processing circuitry 50 may execute monitoring application 72 and in accordance with logic of the executed application, perform a number of operations. In some examples, in response to an indication of the current patient location, processing circuitry 50 transmits, via communication circuitry 60, the datasets of patient data 82 for data submission 86 to computing device(s) 12 and/or computing system(s) 20 of FIG. 1. This transmission may be included in a message indicating the health event, as described herein. Transmission of the message may occur on an ad hoc basis and as quickly as possible as the response to the indication. Communication circuitry 60 may include any suitable hardware, firmware, software, or any combination thereof for wirelessly communicating with another device, such as computing devices 12 and/or IoT devices 30.

Some embodiments of the medical system couple IMD 10 with a remote computing system configured to run one or more computing services including a health monitoring (computing) service. The remote computing system (e.g., computing system(s) 20 of FIG. 1) may communicate packetized data to IMD 10 located within a geofenced geographic area, prompting IMD 10 to execute program code that implements an operation of interest. That packetized data may comprise a message in which the message data identifies the operation of interest. In some examples, the health monitoring service may classify a particular operation as an operation of interest. For instance, operation results may provide information beneficial to patient 4. The health monitoring service may desire the operation results, for example, to improve upon the medical care provided patient 4. In FIG. 1, HMS 22, as an example of the health monitoring service, may be configured to trigger one or more data submissions by computing device 12A. Once triggered, these data submissions may be automatic based on certain conditions such as a state of operation, recency of stored patient data, other functionality of the service, and so forth.

The health monitoring service may invoke functionality to perform an example action that when activated by an indication of current patient location, trigger an upload of recent data. By way of the example action, patient 4 may be directed to put immediate attention to any missed transmissions with the health monitoring service. While in operation, IMD 10 uploads one or more datasets of patient data, but, at times, IMD 10 fails to successfully upload any data. When IMD 10 does not perform a data upload within an expected time frame, that (missed) data upload is one example of a missed transmission. There are other examples of missed transmissions, mainly because IMD 10 may upload desired patient data for a several purposes. This may include a pending transaction that will fail because of an error in IMD 10 or computing device(s) 12.

Monitoring application 72 may be configured to complete data submission 86 by transmitting datasets of patient data 82, in some examples, as a response to being prompted by communications (e.g., messages) from a device of the health monitoring service, such as HMS 22 of FIG. 1, a patient device, such as computing device 12A of FIG. 1, and/or a beacon device, such as IoT device 30 of FIG. 1. A message may indicate a current patient location and if that location corresponds to a geofenced area, monitoring application 72 is configured to automatically transmit patient data in response to requests from a local device (e.g., a beacon device) for the health monitoring service (e.g., HMS 22). As an option, beacon application 76 may be executed in IMD 10 to run as a client or an agent of the health monitoring service.

The health monitoring service may initiate an action that causes beacon application 76 to perform some functionality with monitoring application 72. IMD 10 may receive from the device of the health monitoring service, the patient device, and/or the beacon device a message identifying beacon application 76 as a recipient. Based on contents of the message, beacon application 76 may be directed to transmit an inter-process communication invoking an appropriate function call on monitoring application 72. Receiving the communication may cause monitoring application 72 to perform the initiated action of the health monitoring service. In one example, the function call may be configured to open/start or restart monitoring application 72. In another example, the function call may be configured to transition monitoring application 72 from a sleep state or an active state. In yet another example, the function call may be configured to resume stalled, terminated, and/or dormant operations of monitoring application 72.

To illustrate one example action to initiate via geofencing, the health monitoring service may initiate data submission 86 from IMD 10. A device other than IMD 10 may communicate a message as directed by the health monitoring service. The message may be configured to prompt completion of data submission 86 by having IMD 10 transmit datasets of patient data 82 upon receiving the communicated message. An example corresponding function call on monitoring application 72 may initiate or resume a scheduled/customized transmission of the datasets of patient data 82, completing data submission 86 as directed by the message from the health monitoring service.

In some examples where monitoring application 72 is configured to receive communications over the network from the health monitoring service, monitoring application 72 may be running in a background and in response to at least one communication, transition to running in a foreground. If monitoring application 72 is not running, some communications initiate execution of monitoring application 72. When monitoring application 72 starts running in the foreground, IMD 10 may automatically restart missed transmissions until data submission 86 is complete. If monitoring application 72 enforced a schedule of data uploads, any incomplete upload at the time of the above communications is a missed transaction. The logic for monitoring application 72 may leverage the network connectivity with the computing system of the health monitoring service and immediately handle data submission 86 as packetized data in a response message.

Receiving the above-mentioned communications may cause IMD 10 to complete past or currently due submissions of sensed patient data 82 to the health monitoring service. The health monitoring service may be operative within one or more geographic areas and have its communications with IMD 10 confined therein such that patient 4 entering any one of these areas (i.e., a geofence) with a patient device (e.g., computing device 12A of FIG. 1) causes activation (e.g., a restart) of any missed transmission. The activation may be triggered by IMD 10 receiving an indication of patient 4 entering the geofenced area. The health monitoring service may facilitate other forms of engagement with patient 4 as set forth herein.

As examples, event surveillance 74 may be configured to detect cardiac arrhythmias, worsening heart failure, or other cardiac health events (or simply “cardiac events”) based on an EGM/ECG recording cardiac activity data and/or various physiological parameters indicative the electrical or mechanical activity of heart 6 of patient 4 (FIG. 1). In some examples, event surveillance 74 may detect stroke based on the cardiac activity data. In some examples, sensing circuitry 54 may detect brain activity data, e.g., an electroencephalogram (EEG) via electrodes 56, and event surveillance 74 may detect stroke or a seizure based on the brain activity alone, or in combination with cardiac activity data or other physiological data. When event surveillance 74 detects a health event, event surveillance 74 may store the sensed data 82 that lead to the detection (and in some cases a window of data preceding and/or following the detection) as data submission 86. Prior to transmitting data submission 86, monitoring application 72 may apply an adjudication technique to either confirm or reject the detected health event. Unless rejected by the adjudication technique, monitoring application 72 may generate an alert for patient 4.

The medical system and techniques of the present disclosure may package IMD 10 with external computing resources that results in enhancing IMD 10 in a number of ways. The medical system and techniques descried for FIG. 1 includes external locations with additional storage resources for patient data. The medical system and techniques described for FIG. 1 benefit from engaging (remote) computing services launched by a remote computing system instead of consuming local processing resources for new/updated software components. These capabilities may be new capabilities, firmware updates, support software and other components to improve the operations of IMD 10.

An external device for IMD 10 may include software/hardware components that provide software applications running in IMD 10 with access to storage, processing, and network resources. IMD 10 may use the external device for enhancing functionality of monitoring application 72 running in the external device. FIG. 1 illustrates, as examples of the external device, computing device(s) 12 such as computing device 12A or computing device 12B. When the external device is a mobile device, a mobile application may be configured to facilitate IMD 10 interrogation and patient data assessment process more efficient. By implementing the techniques of the present disclosure, the external device may improve upon the efficiencies of device interrogation and patient data assessment.

Another example external device may be a beacon device, which may be implemented as an IoT device (e.g., IoT device 30 of FIG. 1). The beacon device may be attached to any object and positioned throughout a geofenced area. Via the beacon device, beacon application 76 may be configured to receive beacon communications from the health monitoring service or and/or a server of a local area network. In some examples, the beacon communications are messages, similar to those described herein, that include an indication of a current patient location and/or an invocation of functionality provided by beacon application 76. As directed in an example function call, beacon application 76 may communicate to monitoring application 72 (e.g., as an inter-process communication) the message received from the beacon device if monitoring application is running in the foreground; otherwise, beacon application 76 has to initiate execution of monitoring application 72 and/or transition the executed logic of monitoring application 72 from operating in the background to the foreground. If monitoring application 72 is running in the background, for instance, in sleep mode, beacon application 76 may run a wake-up process with an operating system of IMD 10 to transition monitoring application 72 to the foreground.

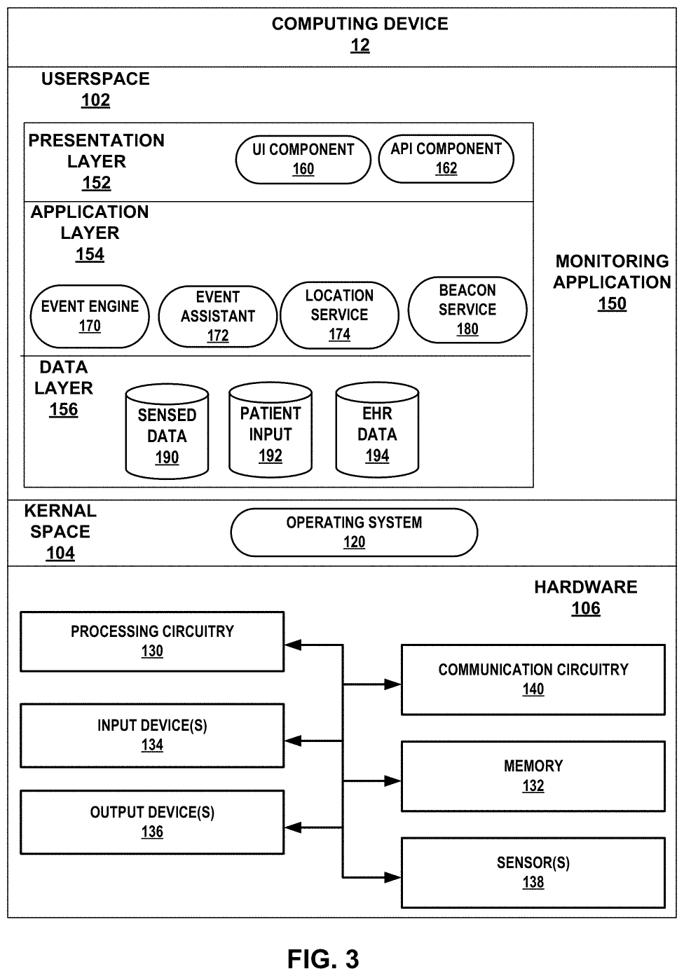

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, IoT devices 30 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 134 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, accelerometers, an optical sensor, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or BLE.

As shown in FIG. 3, monitoring application 150 executes in user space 102 of computing device 12. Monitoring application 150 may be logically divided into presentation layer 152, application layer 154, and data layer 156. Presentation layer 152 may include a user interface (UI) component 160, which generates and renders UI views of health monitoring application 150. Presentation layer 152 may include API component 162, which instantiates an API with functionality for using software modules of application layer 154.

Application layer 154 may include, but is not limited to, an event engine 170, event assistant 172, location service 174, and beacon service 180. Event engine 172 may be responsive to receipt of an indication of a current patient location from a device of HMS 22 or from a different device. Event engine 172 may control performance of any of the operations in response to the indication of the current patient location, particularly, if the current patient location indicates patient 4 entered a geofenced area designed by a medical system. Event engine 172 may be responsive to receipt of a transmission from IMD 10 indicating that IMD 10 detected a health event, e.g., an acute health event.

In some examples, event engine 170 analyzes sensed data 190, and in some examples, patient input 192 and/or EHR data 194, to determine whether there is a sufficient likelihood that patient 4 is experiencing the health event. Sensed data 190 may include data received from IMD 10 as part of a transmission, additional data transmitted from IMD 10, e.g., in “real-time,” and physiological and other data related to the condition of patient 4 collected by computing device(s) 12 and/or IoT devices 30. As examples, sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

A computing system for operating a health monitoring service, HMS 22, may be configured to communicate, over a network, a request for patient input 192 as an example data submission. Health monitoring application 150 may receive the request, generally, in the form of some content for presentation in a user interface (UI) view. In one example, health monitoring application 150 generates the UI view with UI elements configured to receive patient input 192.

Patient input 192 may provide appropriate information for responding to patient forms (e.g., consent forms) and other documents constituting example requests from HMS 22. Patient 4 may complete the necessary consent forms in preparation of a clinic visit and by doing so, patient 4 does not need to spend an early part of the visit completing the consent forms and is provided with medical care for the entire visit.

Patient input 192 may include responses to queries posed by health monitoring application 150 regarding a current condition of patient 4, input by patient 4 or another user, such as clinician 26. The queries and responses may occur responsive to entering a geofenced area and receiving a message from a device of HMS 22, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition, symptoms, or treatments of patient 4 described above. EHR data 194 may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data 194 may also include demographic and other information of patient 4, such as age, gender, height, weight, and BMI.

An assistant 172 may provide a conversational interface for patient 4 and/or clinician 26 to exchange information with computing device 12. Event assistant 176 may query the user regarding the condition of patient 4. Responses from the user may be included as patient input 192. Event assistant 172 may use natural language processing and context data to interpret utterances by the user. In some examples, in addition to receiving responses to queries posed by the assistant, assistant 172 may be configured to respond to queries posed by the user. In some examples, event assistant 172 may provide directions to and respond to queries regarding treatment of patient 4 from patient 4 or clinician 26.

Location service 174 may determine the location of computing device 12 and, thereby, the presumed location of patient 4. Location service 174 may use global position system (GPS) data, multilateration, and/or any other known techniques for locating computing devices.

Beacon service 180 may receive messages from a (local or remote) health monitoring service running in a computing system. The health monitoring service may prompt computing device 12, via monitoring application 150, to perform one or more operations, thereby triggering one or more data submissions to the other computing device for storage. Some data submissions may be considered missed transactions because the monitoring application failed to complete these data submissions for some reason (e.g., being shutdown).

One example missed transmission may be an upload of recent patient data. When beacon service 180 of computing device 12 receives an indication of patient 4's current location being in a geofenced area, beacon service 180 of computing device 12, as a response, may automatically generate output data to upload to the computing system of the health monitoring service, completing the missed transmission of the recent patient data. In one example, beacon service 180 of computing device 12 may be configured to initiate execution of health monitoring application 150 in response to receiving a message from HMS 22. Health monitoring application 150 may be prompted by the received message to complete a missed upload or a pending upload by way of a packetized data transmission, over a network, of the recent patient data to a device within the designated area or from an external location.

In further response to the indication of patient 4's current location, monitoring application 150 may configure computing device 12 for synchronizing sensed patient data and device interrogation data with an external storage location. The patient is a user of a medical device, such as IMD 10 of FIG. 1, which generated the sensed patient data from various signals. In some examples, rules engine 172 may store in memory for event assistant 172 information defining a schedule for future transmissions of the datasets of patient data to the computing system of HMS 22.

API component 162 may include functionality for a health monitoring service (e.g., HMS 22 of FIG. 1) to invoke in order to perform a desired operation. In one example, the computing system of the health monitoring service may communicate a message directed to API component 162. The message may identify a function call, for example, for setting times in the above schedule for transmitting recent datasets of the sensed patient data. In this manner, the health monitoring service may participate in coordinating subsequent uploads to the health monitoring service of the sensed patient data. In effect, following the schedule for future transmissions automates the synchronization between the patient's computing device(s) 12 and the computing system(s) of the health monitoring service.

Location data for the geographic area on which a clinic resides may be programmed into memory of a patient device. Using a number of technologies, the patient device may obtain location data for itself. In one example, a GPS system may provide coordinates for the patient device via a GPS module (e.g., a Global Navigation Satellite System (GNSS) receiver). The patient device may be configured with the GPS module and upon receiving current patient device coordinates, patient device may determine whether the current patient location is within a geofenced area for the clinic. If the current patient device coordinates match a set of geographic coordinates for the clinic, the patient device may initiate one or more data submissions, for example, by way of a function call directed to wake up a monitoring application and invoke application functionality. The function call may execute logic of the monitoring application for performing an interrogation operation to (e.g., automatically) retrieve interrogation data for transmission to a device of the health monitoring service. Hence, receiving the current patient coordinates for patient device (e.g., in a message) causes the patient device to detect the clinical setting and/or the geofenced geographic area, triggering the one or more data submissions by the monitoring application.

Similarly, location data for the geofenced area on which the clinic resides may be programmed into memory of a (remote or local) computing system for running the health monitoring service as a computing service over a network. The network may include the Internet or a local area network. A geofence can be configured for the clinic using the set of geographic coordinates for the geographic area surrounding and/or corresponding to the clinic. Therefore, the location data may include a set of coordinates for the geofenced clinic; the set of coordinates be pre-programmed into the memory (e.g., by the patient), or, alternatively, a location service of the health monitoring service may distribute the set of coordinates as messages sent to subscribers.

The medical systems and techniques may implement geofencing technology to enable the health monitoring service and/or a patient device implementing the application to recognize certain events, such as when the patient visits, for example, a clinical setting. The present disclosure describes examples in which implementing a geofence for a geographic area enhances the operation of the health monitoring service.

A device (e.g., a beacon device) within the geofenced clinic may operate as part of the computing system such that the patient entering the clinic causes the computing system to detect a presence of the patient, triggering one or more data submissions from the patient device. The computing system operating the health monitoring service may be configured using a number of technologies and as such, the device may be configured with a GPS module operative to receive coordinates from the patient device. Based on a comparison between the set of geographic coordinates for the clinic and each iteration of coordinates for the patient device, the computing system of the health monitoring service may determine when the patient is currently located in the clinic and then, responsive to the determination, initiate any data submission that is either past due or due at a clinic appointment time or a current patient time. In some examples, the location data may be an indication (e.g., a message) that a current patient location and the clinic location are within a pre-defined proximity. For example, the indication may be a NFC communication from a NFC module.

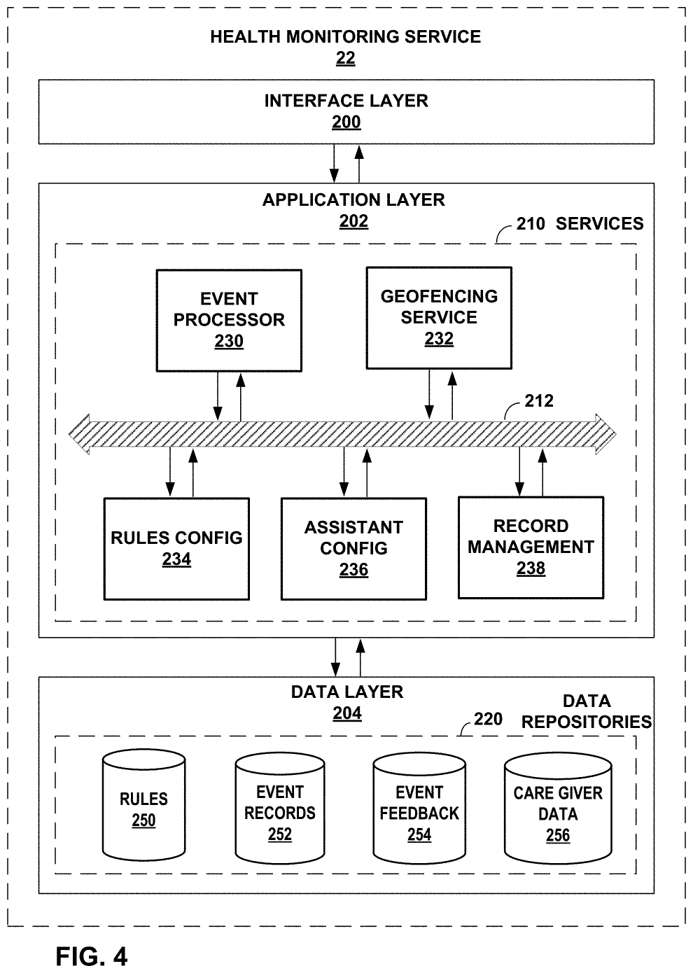

FIG. 4 is a block diagram illustrating an operating perspective of HMS 22. HMS 22 may be implemented in a computing system 20, which may include hardware components such as those of computing device 12, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., an alert of an acute health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from computing device(s) 12 and/or IoT device(s) 30 indicating that IMD 10 detected an acute health event of patient and, in some examples, that the transmitting device confirmed the detection. Event processor service 230 may initiate performance of any of the operations in response to detection of an acute health event ascribed herein to HMS 22, such as communicating with patient 4, clinician 26, and care providers 40, activating drone 46 and, in some cases, analyzing data to confirm or override the detection of the acute health event by IMD 10.

Record management service 238 may store the patient data included in a received alert message within event records 252. Alert service 232 may package the some or all of the data from the event record, in some cases with additional information as described herein, into one more alert messages for transmission to clinician 26 and/or care providers 40. Care giver data 256 may store data used by alert service 232 to identify to whom to send alerts based on locations of potential bystanders 26 and care givers 40 relative to a location of patient 4 and/or applicability of the care provided by care givers 40 to the acute health event experienced by patient 4.

In examples in which HMS 22 performs an analysis to confirm or override the detection of the health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, the Apriori algorithm, K-Means Clustering, k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm acute health event detection, (or in examples in which HMS 22 does not confirm event detection) rules 250 maintained by HMS 22 may include rules 196 utilized by computing devices 12 and rules 84 used by IMD 10. In such examples, rules configuration service 250 may be configured to develop and maintain rules 196 and rules 84. Rules configuration service 234 may be configured to modify these rules based on event feedback data 254 that indicates whether the detections and confirmations of acute health events by IMD 10, computing device 12, and/or HMS 22 were accurate. Event feedback 254 may be received from patient 4, e.g., via computing device(s) 12, or from care providers 40 and/or EHR 24. In some examples, rules configuration service 234 may utilize event records from true and false detections (as indicated by event feedback data 254) and confirmations for supervised machine learning to further train models included as part of rules 250.

As illustrated in the example of FIG. 4, services 210 may also include an assistant configuration service 236 for configuring and interacting with event assistant 176 implemented in computing device 12 or other computing devices.

In some examples, health monitoring service 250 may be active on a different network or a same network as a patient who also is a medical device user. A device of health monitoring service 250 may operate within a geographic area designated for providing medical care to the patient (e.g., a geofenced geographic area). Health monitoring service 250 configures one or more computing systems to be network accessible to the patient's devices, including the medical device and possibly their mobile device(s). The patient may enter the geofenced geographic area and obtain (instantaneous) access to a computing system over a network connection. A variety of actions are enabled for the medical system; the health monitoring service may initiate an action that involves the computing system and ultimately, causes the patient device(s) to perform one or more operations. In some examples, the health monitoring service may desire recent data from the patient device(s) and for that purpose, perform an action to trigger a data upload from the patient device(s).

Over a time period, the patient device(s) senses indicia of patient 4's health and then, generates datasets of sensed patient data. It is advantageous to synchronize those datasets of sensed patient data with the computing system(s) of health monitoring service 250 and to maintain an external storage location with recent and up-to-date information. Health monitoring service may become inoperative or dormant unless recent patient data is available for analysis. There are a number of other causes for inconsistencies in the sensed patient data maintained by the health monitoring service.

The medical systems and techniques may implement geofencing technology to enable health monitoring service 250 to recognize certain events, such as when the patient visits, for example, the clinical setting. Location data for the geographic area on which a clinic resides may be programmed into a computing device at the clinic for the local service such that the patient entering the clinic causes the computing device to identify the patient and trigger one or more data submissions from the patient device(s) (or only the patient). Hence, a geofence can be configured for the clinic using geographic coordinates. The local health monitoring service may be configured using a number of technologies and as such, may be configured with a GPS module operative to receive coordinates of the patient at their current location and/or coordinates of the clinic. Based on a comparison between both sets of coordinates, a device of the local health monitoring service may determine when the patient is currently located in the clinic and then, responsive to the determination, initiate any data submission that is either past due or due at a clinic appointment time or a current patient time. A different embodiment of the location data may be an indication (e.g., a message) that a current patient location and the clinic location are within a pre-defined proximity; one example of the indication may be a NFC communication from a NFC module.

Medical systems and techniques described herein are applicable to computing systems for personal health monitoring of patients without actually being present in a same area and away from a patient's location (i.e., remotely). In summary, medical systems and techniques described herein are capable of reducing the time it takes to interrogate an implantable medical devices, such as an implantable pacemaker, ventricular assist device (VAD), defibrillator, cardiac monitor, or other cardiac device. Improved efficiency and patient flow can be observed with this technology in addition to improved input from device specialists when the patients are remotely located and improved clinical decision making because of the prompt input from specialists.

A clinic may require access to the medical device to retrieve patient data of interest, which may involve having personnel trained for the same manufacturer's devices and equipment (e.g., a programmer module) for programming the medical device as well as another trained person to operate the equipment, in order for medically-trained specialists (e.g., electrophysiologists, cardiologists, device clinic nurses or physician's assistants) to interpret the data and make programming changes when appropriate. This is a physical process for retrieving (e.g., accessing, viewing, and/or transferring) patient data from the patient device(s), and there are inherent delays in such a process, resulting in inefficiencies in patient care. The present disclosure describes medical systems and techniques that are capable of improving efficiencies in a patient's health monitoring. The medical systems and techniques described herein mitigate or eliminate altogether certain inefficiencies in a physical patient data retrieval process, causing a further reduction in (total) latency.

From a clinical application perspective, there are many potential uses for medical systems and techniques described herein. In addition to applying this technology “intra-hospital,” it should also be considered for “inter-hospital” use. The health monitoring service may operate with a network of devices at hospitals and emergency rooms, which are represented in FIG. 1 by facility 38. Once the health monitoring service is established, electrophysiologists are able to initiate the device interrogations remotely. Another application for the techniques described herein is in the device clinic where the medical systems described herein can reduce clinic visit time for patients. Follow-up visits for devices can be very inefficient when multiple patients are waiting in rooms to have their devices checked. Because of the connection to HMS 22 maintained by the geofenced area 28, the patient's medical device can be serviced over network 16 rather than standard programmer checks. Therefore, secure remote monitoring by way of geofencing and remote follow-up as described herein have enhanced the medical care being provided to patient 4. For example, mobile application-based health monitoring as described herein further shifts the clinical follow-up appointments to exception-based assessments.

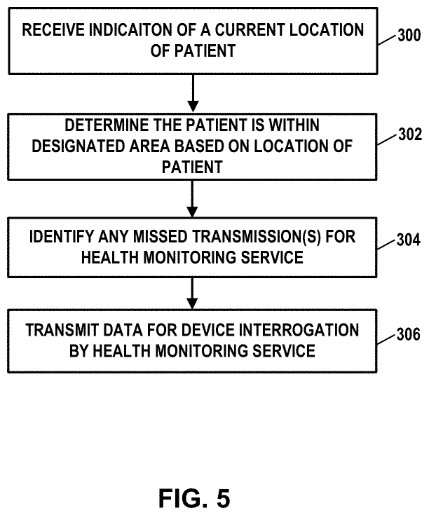

FIG. 5 is a flow diagram illustrating an example operation by a computing device of a medical system that operates in accordance with one or more techniques of the present disclosure. The example operation depicted in FIG. 5 is described with respect to a computing device 12 depicted in FIGS. 1 and 3, but may be described with respect to any one or more devices, e.g., computing devices 12, computing systems 20, IoT devices 30, IMD 10, or health monitoring service (HMS) 22 of FIGS. 1-4 that may implement a health monitoring service and/or client applications of the service. The techniques described herein may be performed by processing circuitry of any one or more of these devices.

According to the illustrated example of FIG. 5, processing circuitry of system 2 (e.g., processing circuitry 130 of one or more computing device(s) 12) provides a computing service for health event monitoring based on a patient's physiological data (e.g., sensed data 190) generated by sensing circuitry 52 of IMD 10 and patient input (e.g., patient input 192). According to the illustrated example of FIG. 5, the processing circuitry detects changes in the patient's health caused by an acute health event such as an arrhythmia or a cardiac arrest.

The present disclosure describes “health monitoring” as a computing service engaging one or more devices to support any given patient with a medical device such as an implantable medical device or a wearable device. Such a patient is likely to benefit from a health monitoring service that expands a patient's present medical care, for instance, beyond the patient's personal devices.

As described above, patients typically rely upon personal devices (e.g., medical devices and mobile devices) for health monitoring outside of a clinical setting. When limited to the patient's personal device(s), the patient cannot benefit from external resources. A patient may accept health monitoring to some degree beyond their (personal) device, such as from an external device over a network; however, the patient's own personal device inhibits such a manner of health monitoring by causing delays in medical device interrogation, inconsistencies in patient data, inefficiencies in patient data assessment and so forth. An external device may be communicatively coupled to the patient's personal device(s) and, via a number of mechanisms, arrange for submissions of patient data and other transmissions from the patient device(s) to either the external device or to another external location.

To commence the health monitoring service of the example operation of FIG. 5, the processing circuitry receives an indication of a current location of a patient (300). The indication may be a set of GPS coordinates provided by a GPS transceiver in one of the patient device(s). In another example, the indication may be a message notifying the patient of a geofenced geographic area designated for medical care of the patient (e.g., a clinical setting). In some examples, a computing system may run a computing service for the geofenced geographic area and communicate a message to the patient device in response to the patient's presence in that area. As an alternative, the indication may notify the patient of their proximity to the geofenced geographic area. The message may be arranged into packetized data in accordance with any communication technology (e.g., protocol).

The computing system of the geofenced geographic area may avail a low energy wireless communication technology, such as BLE, as the underlying protocol for communicating a message indicative of the current location of a patient. In some examples, the indication may be in the form of a beacon message (e.g., advertisement) that a BLE-enabled beacon device broadcasts at a regular interval. The computing system may operate as a beacon device and/or configure one or more beacon devices to operate within the geofenced geographic area.

The patient device, via signals (e.g., radio waves), receives the beacon message and based on message content, proceeds to determine that their current location is within the geofenced geographic area. The patient device may determine that the beacon message is directed towards an application and configured to trigger actions by the patient device. The computing system of the geofenced geographic area, operating as an iBeacon device in accordance with Apple iBeacon technology, may communicate an iBeacon advertisement message, as one example embodiment of the beacon message, to any device in that area. In turn, the patient device, while listening for signals from iBeacon devices, senses the iBeacon device operated by the computing system by capturing the iBeacon advertisement message. and then, in response to the advertisement, communicating an acknowledgement of a connection with the computing system running the computing service for the geofenced geographic area. The connection with the computing system allows the computing service to provide secure and hyper-contextual content (e.g., formal documents).

In the example of FIG. 5, the processing circuitry determines the patient is within a geofenced geographic area (e.g., area 28 of FIG. 1) based on the current location of the patient (302). Based on the determination, the patient device(s) may recognize the geofenced geographic area as an environment in which engagement by the patient with the health monitoring service is facilitated and secure. The connection with the computing system running the service further facilitates and secures the methods of patient engagement, permitting submissions of patient data and other communications with the health monitoring service, which are protected from adversarial intrusion (e.g., from misappropriation or disclosure).

The health monitoring service, in general, detects changes in the health of patients based upon patient data. For example, by way of the above-mentioned patient engagement, the computing system running the health monitoring service may perform an assessment of any patient with as pacemakers, defibrillators, monitors, and other examples of implantable medical device(s) (e.g., IMD 10 of FIG. 1). The health monitoring service may configure the computing system to operate as a beacon device and advantageously employ beacon messages to trigger application logic on the patient device.

The patient device may be a mobile device configured to receive beacon messages and in response, invoke appropriate functionality for each corresponding application. As described herein, a monitoring application running in the mobile device may be a client application for the health monitoring service and to that end, provide the computing system with various patient data generated by the medical device. If inoperable, however, the monitoring application may fail to transmit any patient data despite being requested and/or scheduled to do so. The processing circuitry may identify missed transmissions for the health monitoring service (304). These transmissions may be uploads of recent patient that have failed and/or are pending.

Without patient involvement, the processing circuitry of the patient device may be unable to restart or complete failed data submissions or may consume substantial quantities of resources to do so. The medical systems and techniques described herein dedicate the health computing service for such restarting/triggering. Having an API to implement functionality for the health computing service to use allows another computing device (and another user) to initiate automatic transmissions of certain datasets of patient data. The health monitoring service may benefit from having the recent patient data and therefore, may request automatic transmissions of that data, for example, when performing a device interrogation of the patient's medical device. In response, the patient device(s) may initiate the requested transmissions to provide external computing systems with datasets of the most recent patient data, including sensed patient data generated by the patient's medical device, in performance of the device interrogation by the health monitoring service (306).

Furthermore, the medical systems and techniques described herein may implement geofencing technology for the health monitoring service, which operates on a local and/or remote computing system and is configured to recognize certain events, such as when the patient visits, for example, the clinical setting. Location data for the geographic area on which a clinic resides may be programmed into a computing device at the clinic for the local service such that the patient entering the clinic causes the computing device to identify the patient and trigger one or more data submissions from the patient device(s) (or only the patient). Hence, a geofence can be configured for the clinic using geographic coordinates. The local health monitoring service may be configured using a number of technologies and as such, may be configured with a GPS module operative to receive coordinates of the patient at their current location and/or coordinates of the clinic. Based on a comparison between both sets of coordinates, a device of the local health monitoring service may determine when the patient is currently located in the clinic and then, responsive to the determination, initiate any data submission that is either past due or due at a clinic appointment time or a current patient time. A different embodiment of the location data may be an indication (e.g., a message) that a current patient location and the clinic location are within a pre-defined proximity; one example of the indication may be a NFC communication from a NFC module.

To illustrate by way of example, the medical system and techniques are configured to monitor the device interrogation and identify instances whether the device interrogation has been interrupted for any reason. In addition to datasets of patient data that are collected and stored by the medical device, the monitoring application may collect and store datasets of other patient data in the patient's mobile device, and then submit both datasets for the interrupted interrogation. By mitigating such interruptions and keeping the monitoring application active, the medical system and techniques may achieve low latencies in an amount of elapsed time between a capture of a dataset and a transmission of that dataset for external (e.g., remote) storage.

To save additional time while the patient is present in the geofenced area, the health monitoring service may initiate preparation and submission of hospital admission documents (e.g., consent forms or health questionnaires) on the patient's behalf, reducing the typical delays associated with hospital admission, clinic visitation (e.g., follow-up visitation), and/or the like. The patient may complete the necessary consent forms and intake documents while at home or in the waiting room/lobby. These consent forms are one example embodiment of a data submission for the health monitoring service; any document conveying patient information in some format may be presented to clinicians via computing device 12A. It is possible for the health monitoring service to populate such documents with appropriate patient data (e.g., name, address, age, health insurance information, malady, etc.). A patient history, another example data submission for the health monitoring service, may be requested and/or prepared for the patient. A journal, an example patient history, may arrange information memorializing symptoms that are recorded by the patient or the medical device. The patient may be prompted to provide and submit self-written symptom journals, or have their self-written symptom journals automatically submitted from their patient devices without patient involvement, in response to entering the geofenced area. The patient device may generate, in response to a request for a patient history, a journal memorializing symptoms to include in output data for a packetized data transmission to a device of the computing system running the health monitoring service (e.g., the local health monitoring service).

As another example, the computing system running the health monitoring service may be instantly notified of device malfunctions in addition to receiving secure notifications regarding health event detections (e.g., cardiac arrhythmias), confidential notifications, and other private data. These notifications are secured by virtue of the service having control over the geofenced area.

Having the patient's physical presence allows the clinical setting to administer the consent documents and obtain informed consent without communicating any data outside the clinical setting (if desired). It is possible for the clinic to configure the local computing device to obtain informed consent without electronic means or a trivial level of electronic means. The patient may sign and submit a paper copy (without receiving a copy via email), complete an electronic form displayed by the computing device at the clinic, submit an electronic copy via Bluetooth® telemetry, and/or the like.

As the patient enters the clinic, the computing device of the local service may recognize the patient as a service user and determine that device interrogation halted or stalled. This occurs, for example, when the patient's mobile device (via a monitoring application) and/or the patient's medical device ceases scheduled transmissions and/or fails to answer requests for (customized) transmissions of patient data. Recent measurements and/or updated readings of patient physiological parameters have not been uploaded. Without the monitoring application running in the patient device, the patient's consent forms cannot be downloaded. There are a number of other examples of device interrogation interruption. Unable to retrieve recent patient data, the computing device at the clinic cannot complete an assessment regarding patient health.

If the patient device is shutdown and/or the monitoring application is disabled or closed, the computing device initiate a process (e.g., a wake up process) to load into memory and execute logic of the monitoring application to run a main process and possibly, additional processes. The main process of the monitoring application performs a number of operations including at least one data submission of patient data from the patient device to the computing device of the clinic. The medical system may implement functionality on an API for prompting the main process to perform an operation, such as a transmission of the patient data for a scheduled data submission that is past due. The computing device at the clinic may invoke a function call to trigger (e.g., periodic) transmissions of patient data to facilitate a pending clinic appointment. If the local health monitoring service previously submitted a request for specific datasets of patient data and that request was not filled by the monitoring application, the computing device at the clinic may invoke a function call to trigger a transmission of a requested data submission that is past due otherwise late. Instead of disabled or closed, the monitoring application may be dormant, for example, where the main process is running on processing circuitry but restricted to a background according to another example. In yet another example, the main process may have raised a fault and is not respond to any inter-process communications.

A remote health monitoring service may be configured as a computing service (e.g., a cloud service) that is accessible over a network from any geographic area using any number of communication protocols as described herein such as the Internet. The remote health monitoring service may combine a local health monitoring service with a network service (e.g., a cloud service) running on a network-accessible resource and built using network functions that are compatible with any number of protocols. The network-accessible resource at the external location may be used to store patient data and facilitate the patient's medical care. Similar to the local health monitoring service, the remote health monitoring service may initiate a main process of the monitoring application to performs a number of operations including at least one data submission of patient data from the patient device to the computing device of the clinic and/or the storage resources of the external location. Each data submission directed towards the remote health monitoring service is packetized into data units of a relevant protocol.

The order and flow of the operation illustrated in FIG. 5 are examples. In other examples according to this disclosure, more or fewer thresholds may be considered. Further, in some examples, processing circuitry may perform or not perform the methods of FIG. 5, or any of the techniques described herein, as directed by a user, e.g., via external device 12 or computing devices 100. For example, a patient, clinician, or other user may turn on or off functionality for identifying changes in patient health (e.g., using Wi-Fi or cellular services) or locally (e.g., using an application provided on a patient's cellular phone or using a medical device programmer).

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. A method comprising:
determining, by processing circuitry, that a current patient location corresponds to a designated area of a medical system in response to receiving, by an input device, input data comprising an indication of the current patient location;
in response to the determination, generating, by the processing circuitry, output data comprising one or more datasets of patient data for a data submission to a health monitoring service, wherein the patient data is stored in memory for a patient having a medical device configured to generate at least a portion of the patient data; and
transmitting, by an output device, to the health monitoring service, the output data to complete the data submission.

2. The method of claim 1, wherein determining that the current patient location corresponds to the designated area of the medical system further comprises:
initiating an active operating mode for a monitoring application, wherein the monitoring application is to run in a foreground of a patient device.

3. The method of claim 1, wherein a device of the health monitoring service communicates the indication in a message, wherein the health monitoring service is operating on a computing system within the designated area or on a computing system of an external location to the designated area.

4. The method of claim 1, wherein a device of the health monitoring service communicates the indication in a message, wherein the health monitoring services is operating on a computing system comprising at least one of a local computing system or a remote computing system.

5. The method of claim 1, wherein determining that the current patient location corresponds to the designated area of the medical system further comprises:
determining that the current patient location corresponds to the designated area of the medical system based upon receiving a message from a device of the health monitoring service operating on the computing system within the designated area.

6. The method of claim 1, wherein determining that the current patient location corresponds to the designated area of the medical system further comprises:
communicating, by the output device, an acknowledgement to a device of the health monitoring service in response to a connection request from that device.

7. The method of claim 1, wherein determining that the current patient location corresponds to the designated area of the medical system further comprises:
in response to the indication of the current patient location from a location service, initiating a function call to trigger the transmitting to the health monitoring service.

8. The method of claim 1, wherein determining that the current patient location corresponds to the designated area of the medical system further comprises:
in response to a function call, triggering transmission, by a monitoring application, of the one or more datasets of the patient data to the health monitoring service, wherein the triggering comprises executing logic for the monitoring application, wherein a second application initiates the function call based on the indication of the current patient location, wherein the function call causes the monitoring application to run on the processing circuitry.

9. The method of claim 1, wherein a location service for the health monitoring system communicates, over a network, the indication to prompt a monitoring application to identify, for the data submission, a missed transmission or a pending transmission of interrogation data of the medical device.

10. The method of claim 1, wherein a programmable beacon communicates the indication to prompt a monitoring application to identify, for the data submission, a missed transmission or a pending transmission of interrogation data of the medical device.

11. The method of claim 1, wherein a global positioning system communicates, over a network, the indication to prompt a monitoring application to identify, for the data submission, a missed transmission or a pending transmission of interrogation data of the medical device.

12. The method of claim 1, wherein a computing system for operating the health monitoring service communicates, over a network, a request for patient input as the data submission, wherein the request comprises content for presentation in a user interface (UI) view, wherein the UI view comprises UI elements configured to receive the patient input.

13. The method of claim 1, wherein generating, by the processing circuitry, the output data further comprises:
in accordance with a request for patient input, generating the output data comprising patient responses to queries provided in the request, wherein the request comprises content for presentation in a user interface (UI) view, wherein the UI view comprises UI elements configured to receive the patient input, wherein each UI element is rendered to present the content for a corresponding query and an input control for the patient to submit a patient response for that query.

14. The method of claim 1, wherein generating, by the processing circuitry, the output data further comprises:
generating the output data comprising a patient history as the data submission for the health monitoring service, wherein the patient history comprises a journal memorializing symptoms that are recorded by the patient or the medical device.

15. The method of claim 1, wherein generating, by the processing circuitry, the output data further comprises:
in response to a request for a patient history, generating the output data comprising a journal memorializing symptoms that are recorded by the patient or the medical device.

16. The method of claim 1, wherein generating, by the processing circuitry, the output data further comprises:
in accordance with a schedule stored in the memory, generating the output data comprising the one or more datasets of the patient data for the data submission that corresponds to a scheduled timeslot before a current time.

17. The method of claim 1, wherein generating, by the processing circuitry, the output data comprises:
in response to identifying a request that is past due to the computing system operating the health monitoring service, generating the output data comprising the one or more datasets of the patient data corresponding to the past due request.

18. The method of claim 1, wherein generating, by the processing circuitry, the output data comprises:
in response to receiving a request from the computing system operating the health monitoring service, generating the output data comprising the one or more datasets of the patient data for the data submission that corresponds to the request received from the device.

19. The method of claim 1, wherein generating, by the processing circuitry, the output data comprises:
responsive to an alarm generated by the medical device for the patient, generating the output data comprising the one or more datasets of the patient data for the data submission that indicates a health alert for the patient.

20. The method of claim 1, wherein generating, by the processing circuitry, the output data comprises:
responsive to an alarm generated by the medical device for the patient, determining that a health event corresponding to the alarm is a false positive based on one or more detection criteria; and
withholding transmission of the output data comprising the health event.

21. The method of claim 1 further comprising:
responsive to an alarm generated by the medical device for the patient, communicating, between a device of the health monitoring service and a patient device, a cancellation of the alarm based on one or more cancellation criteria.

22. A medical system comprising:
processing circuitry executing logic for a monitoring application stored in memory configured to:
determine that a current patient location corresponds to a designated area of a medical system in response to receiving, by an input device, input data comprising an indication of the current patient location;
in response to the indication, generate output data comprising one or more datasets of patient data for a data submission to a health monitoring service, wherein the patient data is stored in memory for a patient having a medical device configured to generate at least a portion of the patient data; and
by an output device, transmit, to the health monitoring service, the output data to complete the data submission.

23. The medical system of claim 22, wherein a patient device comprises the input device, the output device, and the processing circuitry.

24. The medical system of claim 22 further comprising communication circuitry that includes a BLUETOOTH module communicatively coupled to the medical device and configured to store timestamped one or more datasets of the patient data in response to one or more data downloads from the medical device.

25. A medical system comprising:
processing circuitry executing logic stored in memory configured to:
detect a presence of a patient device within a designated area based on a broadcast message of the patient device, wherein the patient device comprises a monitoring application configured to upload, to a health monitoring service operating on a computing system within the designated area of the medical system or from an external location of the medical system, one or more datasets of patient data, wherein a portion of the patient data comprises interrogation data of a medical device associated with a patient;
communicate a message comprising an indication of a current patient location, wherein the message is configured to trigger one or more automatic data submissions by the monitoring application as a response to receiving the message; and
receive, from the patient device, the one or more automatic data submissions for the health monitoring service.

26. The medical system of claim 25, wherein the patient device is to initiate execution of the monitoring application in response to receiving the message, wherein the monitoring application is prompted to complete a missed upload or a pending upload by way of a transmission, over a network, of recent patient data from the patient device to a device within the designated area or from an external location.

27. The medical system of claim 25, wherein the message is directed to a second application that is different from the monitoring application, wherein the second application is configured to invoke a function call causing the monitoring application to transmit the one or more datasets of the patient data in completion of the one or more automatic data submissions to a device of the health monitoring system.

28. The medical system of claim 25, wherein communicating the message further comprises:
communicating, to the patient device, a connection request to the patient device; and
receiving, from the patient device, an acknowledgement of the connection request.

29. The medical system of claim 25 further comprising:
communicating, to the patient device, a request for physiological information of the patient for the health monitoring service.

30. The medical system of claim 25 further comprising a device of the health monitoring service communicatively coupled to the patient device and comprising the processing circuitry.

31. The medical system of claim 25, wherein a device operative within the designated area as an intermediate between the patient device and the health monitoring service comprises the processing circuitry.

32. The medical system of claim 25, wherein the medical device is communicatively coupled to the patient device, wherein the medical device comprises at least one of an implantable device, a wearable device, a pacemaker, a defibrillator, or a ventricular assist device (VAD) that comprises one or more sensors and sensing circuitry.

33. A non-transitory computer-readable storage medium comprising program instructions that, when executed by processing circuitry of a medical device, cause the processing circuitry to:
determine that a current patient location corresponds to a designated area of a medical system in response to receiving, by an input device, input data comprising an indication of the current patient location;
in response to the indication, generate output data comprising one or more datasets of patient data for a data submission to a health monitoring service, wherein the patient data is stored in memory for a patient having a medical device configured to generate at least a portion of the patient data; and
by an output device, transmit, to the health monitoring service, the output data to complete the data submission.
